(19) United States
(12) Patent Application Publication
(10) Pub. No.: US 2020/0261229 A1
(43) Pub. Date: Aug. 20, 2020

(54) A SURGICAL PROCEDURE FOR CANCEROUS MANDIBULAR RECONSTRUCTION AND A TEMPORARY MANDIBULAR SPACER THEREFOR

(71) Applicant: TMJ ORTHOPAEDICS PTY LTD, East Melbourne (AU)

(72) Inventor: George DIMITROULIS, East Melbourne (AU)

(21) Appl. No.: 16/648,395

(22) PCT Filed: Sep. 18, 2018
(86) PCT No.: PCT/AU2018/051018

§ 371 Date: Mar. 18, 2020

(30) Foreign Application Priority Data
Sep. 21, 2017 (AU) 2017903841

(51) Int. Cl. A61F 2/28; A61B 17/15

(52) U.S. Cl. CPC A61F 2/2803; A61B 2017/00915; A61B 17/15

(57) ABSTRACT

There is provided herein a surgical procedure for cancerous mandibular reconstruction and a temporary mandibular spacer therefor. The spacer is a structural load-bearing and rigid/stiff interim space maintenance device that can be used by surgeons to temporarily bridge the gap across a resected part of the mandible. The device may be made of a rigid and biocompatible polymer material which is anatomically shaped like a natural mandible. During surgery, the mandibular spacer may be quickly cut with a surgical saw into the desired lengths by the surgeon which may more precisely match the resected segment of native mandible. The length and the shapes of the terminal ends of the spacer may be configured to more precisely maintain the position of the remaining mandibular segments in their original locations and thus preserve the occlusion and surrounding soft tissues in their correct anatomical positions, reducing or eliminating scarring and/or malocclusion.

### FIELD OF THE INVENTION

This invention relates generally to cancerous mandibular reconstruction. More particularly, this invention relates to surgical procedures for cancerous mandibular reconstruction and a temporary mandibular spacer therefor.

### BACKGROUND OF THE INVENTION

Oral cancer makes up 2-3% of all cancers in the Western world and up to 40% of cancers in the Indian subcontinent. Patients with oral cancer are often managed with surgery or radiation therapy, while more advanced cases may also require chemotherapy.

Surgical management of oral cancer occasionally involves the resection of the mandible which may result in severe aesthetic and functional deficiencies if the jaw bone and surrounding soft tissue is not adequately reconstructed. Speech, swallowing, airway, chewing and saliva control are all potentially compromised when segments of the mandible are surgically removed to control the spread of malignant disease. The resultant defect could also lead to scarring and contraction of surrounding tissues which leads to malocclusion due to muscle pull that can also adversely affect tongue attachment and function.

Reconstruction of the mandible may either be done at the primary surgery or delayed and undertaken as a secondary procedure. Currently, the majority of cancer units around the world undertake mandibular reconstruction at the same time as the primary resection in order to avoid scarring and displacement of the remaining mandibular segments. However, mandibular reconstruction at the time of the primary resection complicates any secondary reconstruction.

The current “gold standard” for mandibular reconstruction at the time of primary resection the is vascularised free flap which however requires an additional procedure to harvest of tissue from another part of the body. Other methods include autogenous bone blocks, bridging plates, cancellous bone in titanium mesh, tissue engineered bone scaffold and patient specific alloplastic replacement.

Unfortunately, primary mandibular reconstruction often means many additional hours of complex surgery beyond the resection phase which often results in poorly positioned free flaps and bone grafts that are literally plugged into position to fill the hard and soft tissue gaps. Furthermore, many patients have an unknown prognosis and the surgical resection margins may not be adequately managed, requiring adjunctive radiation therapy. Yet further, cancer treatment may damage autogenous implants and radiation therapy may be hindered by non-radiolucent metallic implants.

US 2005/0273165 A1 (GRIFFITHS et al.) 8 Dec. 2005 [hereinafter referred to as D1] discloses a temporary orthopedic tissue spacer for reconstruction at the time of resection which is replaced with a more permanent implant during revision surgery. The spacer of D1 is flexible to allow the surgeon to bend and squeeze the spacer to fit the resection.

However, with reference to paragraph 59 of D1, the spacer of D1 is not intended to be a load-bearing structural element on account of the shape and size adjustable flexibility thereof and which therefore may cause malocclusion unless it is reinforced with bone plates extending across the spacer which further complicates the primary surgery and may interfere with radiation therapy.

Other alloplastic replacements for mandibular reconstruction may be designed based on patient anatomy. The process of acquiring a patient scan, model designing, manufacturing, and surgery is likely to be costly in terms of time, during which, malignant pathology may have metastasized and would require a new prosthetic as the region of resection would have changed.

The present invention seeks to provide a way to overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE DISCLOSURE

There is provided herein a surgical procedure for cancerous mandibular reconstruction and a temporary mandibular spacer therefor.

The temporary mandibular spacer is a structural load-bearing and rigid/stiff interim space maintenance device that can be used by surgeons to temporarily bridge the gap across a resected part of the mandible.

The device may be made of a rigid and biocompatible polymer material which is anatomically shaped like a natural mandible.

During surgery, the mandibular spacer may be quickly cut with a surgical saw into the desired lengths by the surgeon which may more precisely match the resected segment of native mandible. The length and the shapes of the terminal ends of the spacer may be configured to more precisely maintain the position of the remaining mandibular segments in their original locations and thus preserve the occlusion and surrounding soft tissues in their correct anatomical positions, reducing or eliminating scarring and/or malocclusion as may prior art procedures.

The present spacer is furthermore non-autogenous, thereby allowing for cancer treatment including by way of chemotherapy and is furthermore preferably radiolucent allowing for radiation therapy without interference thereof.

The spacer is left in situ for about 6 to 12 months which gives surgeons the opportunity to reassess the patient's prognosis and provide adequate time for more sophisticated planning of the definitive mandibular reconstruction, either using microvascular composite free flaps or other alloplastic devices.

If the patient survives, the patient may return in about 6-12 months for definitive mandibular reconstruction where the temporary mandibular spacer is easily removed from its tissue bed and the cavity is filled with autogenous vascularized bone or other alloplastic material.

The spacer is preferably manufactured in a hard polymer that is biocompatible and can be sterilized without affecting the physical properties of the material. The spacer is also preferably shaped as a full human mandible without the teeth and coronoid process. The mandibular spacer may come in variety of anatomical shapes and sizes that accurately reflect actual human mandibles. For example, we modelled one spacer from over 100 CT scans that were collated at the University of Melbourne.

The mandibular spacer may be delivered sterilized and packaged to the surgery as a whole mandible. The surgeon may select the most appropriately sized spacer and use a reciprocating surgical saw blade to quickly and easily cut the spacer to the desired lengths that correspond to the actual resected mandibular segment.

In embodiments, the mandibular spacer may comprise cutting guides which may take the form of anatomically placed grooves corresponding to the most common resection margins which guide the surgeon in cutting up the various segment lengths of the spacer that more accurately match the actual resected jaw bone. The grooves make guide the cutting tool during cutting.

The cutting guides allow for the more accurate sizing and shaping of the mandibular spacer portion which enhances the structural interface with the remaining mandibular sections. Furthermore, the cutting guides may simplify the operation in reducing or eliminating “trial fit” iterations as, for example, as alluded to in paragraph 63 of D1 which doesn't have such anatomically placed cutting guides.

In embodiments, the spacer may be cut with reference to offset and/or angle adjustments which may even furthermore precisely size and configure the distal ends of the cut spacer section for better structural securement. Offset and/or angle adjustment may be configured during computer aided modelling and used to configure the cutting guides, generate cutting guide references or be used with reference to the existing cutting guides to make adjusted cuts.

The cut spacer segment is then positioned across the resected segment of mandible and may be fixed to the remaining natural mandibular segments with bone plates and screws.

The temporary mandibular spacer thus facilitates mandibular reconstruction by anatomically replacing the missing jaw segment in a simple and relatively quick procedure that may require only very basic surgical skills.

By utilizing the temporary mandibular spacer, the duration, morbidity and complexity of the primary surgery may be significantly reduced so that the patient's recovery and discharge from hospital is much quicker. Furthermore, the radiolucent nature of the polymer means the mandibular spacer does not interfere with subsequent radiation therapy to the affected area.

The temporary mandibular spacer may also afford surgeons ample time to plan the definitive reconstruction surgery up to 12 months later by providing a solid, three dimensional and anatomically accurate scaffold that ensures the remaining hard and soft tissues are properly anchored in their correct locations. This means that the primary cancer surgery is not delayed because of the need to plan the reconstructive surgery. It also means the original mandibular anatomy is preserved which helps simplify secondary reconstructive surgery, which is often plagued by scarring and malpositioning of the remaining jaw segments.

Furthermore, the mandibular spacer is readily available in various stock sizes and shapes that can be easily predetermined on basic diagnostic CT scans and delivered to the hospital, ready for immediate use.

The mandibular spacer can also be used in salvage operations for patients with terminal prognosis, which provides the patient with a significantly improved quality of life without the need for complex and unnecessarily prolonged surgery.

Presently, the market lacks a product that matches the fundamental characteristics of the present mandibular spacer. Surgeons, particularly in countries with limited surgical expertise and resources rely on heavy metal bone plates to maintain the resected gap that are difficult to contour and secure and often fracture within a few weeks of installation.

Furthermore, unlike the spacer disclosed in D1 which is flexible for size and shape tolerance, the present spacer is substantially rigid so as to be substantially load-bearing, preventing or reducing malocclusion without the need for the metallic bridging plates of D1 which may complicate the primary surgery and interfere with radiation therapy.

The temporary mandibular spacer, by virtue of its anatomical shape, biocompatibility, strength and ease of cutting into a multitude of lengths, and ease of fixation to adjacent natural bone, may become an indispensable device in the surgical management of oral cancer patients who lose part of their mandible to malignant disease.

According to one aspect, there is provided, a surgical procedure for cancerous mandibular reconstruction, the procedure comprising: a primary resection and temporary reconstruction phase comprising: obtaining a temporary mandibular spacer, the spacer having a rigid, biocompatible and radiolucent polymer material body being of a generic and whole mandibular shape; resection of a cancerous section from a mandible of a patient; cutting the temporary mandibular spacer according to the length and position of the resected section to obtain a temporary mandibular spacer portion; inserting the mandibular spacer portion into the resected section and interfacing the opposite ends of the spacer portion to respective ends of the mandible adjacent the resected section to more precisely maintain the position and locations of adjacent remaining mandibular segments; closing surrounding soft tissue to cover the temporary mandibular spacer portion; and a secondary revision reconstruction phase performed a substantial time after the primary resection and temporary reconstruction phase, the revision reconstruction phase comprising: opening the surrounding soft tissue; removal of the temporary mandibular spacer portion; and secondary mandibular reconstruction.

The whole mandibular shape may comprise an elongate body portion curved through a midline symphysis location and terminating at a posterior upwardly extending ramus process portions.

The upwardly extending ramus process portions may terminate in superior condylar process portions.

The temporary mandibular spacer may comprise a plurality of cutting guides and wherein cutting the temporary mandibular spacer may comprise cutting the mandibular spacer with reference to the cutting guides.

The cutting guides may comprise at least one cutting guide being orthogonal with an elongate axis of the elongate body portion.

The cutting guides may comprise at least one cutting guide being angled at an angle with respect to an elongate axis of the elongate body portion.

The angle is angled with reference to the gonial angle.

The cutting guides may be planar intersection cutting guides.

The cutting guides may be located according to reference positions comprising at least one of a midline symphysis location, parasymphysis locations and tooth locations.

The cutting guides may be scored into a surface of the spacer.

The cutting guides have a width of approximately 2 mm.

The secondary mandibular reconstruction may comprise mandibular reconstruction comprising insertion of at least one of autogenous vascularized bone and other alloplastic material.

The secondary revision reconstruction phase is performed more than 4 months after the primary resection and temporary reconstruction phase.

The secondary revision reconstruction phase is performed more than 6 months after the primary resection and temporary reconstruction phase.

The secondary revision reconstruction phase is performed more than 12 months after the primary resection and temporary reconstruction phase.

The material is autoclave compatible.

The material is a polymer.

The material may comprise Polyether ether ketone (PEEK).

The method may comprise providing a plurality of generically shaped spacers of different sizes and wherein obtaining the spacer may comprise selection of an appropriate size according to patient sizing.

The generic mandibular shape is derived from a human mandibles anatomical survey.

The generically shaped spacers may be each classified according to at least one of age, gender and ethnicity according to the anatomical survey.

Connecting the opposite ends of the spacer portion to the respective ends of the resected mandible may comprise bone plate and screw affixation.

The procedure further may comprise radiation therapy prior the secondary revision reconstruction phase.

The procedure further may comprise cutting the temporary mandibular spacer according to reference at least one of offset and angle adjustments.

The least one of offset and angle adjustments may be made with reference to at least of size and position of a diseased portion of the mandible.

The procedure further may comprise computer aided modelling comprising display of a model of the temporary mandibular spacer and user interface control thereon for configuration of the at least one of the offset and the angle adjustments.

The user interface controls may comprise at least one of offset and angle adjustment controls.

The further may comprise forming the cutting guides on the spacer with reference to the at least one of the offset and angle adjustments.

The surgical procedure may further may comprise cutting the temporary mandibular spacer with reference to a cutting guide and the at least one of the offset and angle adjustments.

The procedure further comprises making a cutting guide reference configured according to the at least one of the offset and angle adjustments and cutting the temporary mandibular spacer using the cutting guide.

The cutting guide reference may reference at least one of geometry, cutting guides and reference engagements of the spacer.

The cutting guide reference may comprise a cutting guide face for guiding a cutting tool.

According to another aspect, there is provided a temporary mandibular spacer, the spacer having a rigid, biocompatible and radiolucent polymer material being of generic and whole mandibular shape.

The temporary mandibular spacer may further may comprise a plurality of cutting guides.

The cutting guides may be planar intersection cutting guides.

The cutting guides may be located at anatomically placed resection margins.

The resection margins may comprise at least one of midline symphysis, parasymphysis and gonial angle margins.

The temporary mandibular spacer is obtained from anatomical survey data obtained from a plurality of patients.

The temporary mandibular spacer is classified according to at least one of age, gender and ethnicity according to the anatomical survey data.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a perspective shaded view of the mandible spacer in accordance with an embodiment;

FIG. 2 shows a front elevation view of the spacer;

FIG. 3 shows a top plan view of the spacer;

FIG. 4 shows a side elevation view of the spacer;

FIG. 5 shows a procedure for mandibular reconstruction in accordance with an embodiment; and

FIG. 6 shows a procedure for adjusting cutting of the mandibular spacer portion in accordance with an embodiment.

### DESCRIPTION OF EMBODIMENTS

A temporary mandibular spacer 1 is a rigid, stiff, biocompatible and preferably radiolucent polymer material body, such as one made from Polyether ether ketone (PEEK).

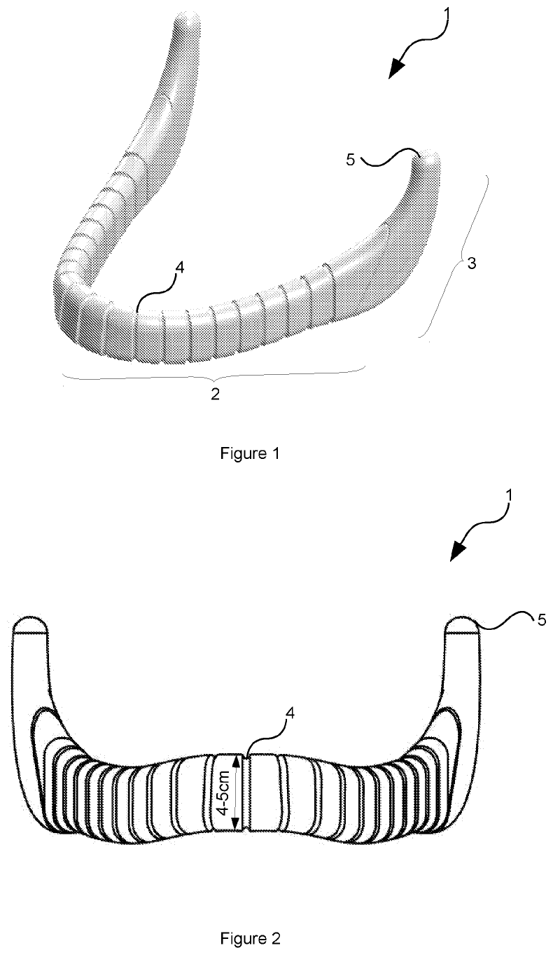

With reference to FIG. 1, the spacer 1 comprises a substantially whole mandible shape comprising a body portion 2 that transitions through the midline symphysis and has posterior upwardly extending ramus portions 3 each terminating at a condylar process portion 5.

The body portion 2 of the spacer is generally elongate and substantially ovular or rounded rectangular in cross-section.

The spacer 1 may be of non-patient specific generic shape and derived from averages obtained from an anatomical CAT scan survey of a plurality of patients. A plurality of spacers 1 of differing sizes may be provided prepackaged and sterilised from which one may be selected according to the sizing of the patient.

The spacer 1 may comprise a plurality of cutting guides 4 for guiding cutting in use.

The cutting guides 4 may be marked on the spacer 1 or, in the embodiment shown, scored by way of grooves into the surface thereof. The grooves may guide the cutting implement such as a reciprocating saw and may, for example, be approximately 2 mm wide in this regard. It should be noted that the cutting guides 4 are not deep enough to compromise the structural rigidity of the spacer 1 such that the spacer 1 yet remain structurally rigid. In this regard, the scored cutting guides 4 may not extend substantially more than 1 mm into the surface of the spacer 1 in embodiments.

In embodiments, the cutting guides are generally planar intersection cutting guides such that one can cut straight through the spacer 1.

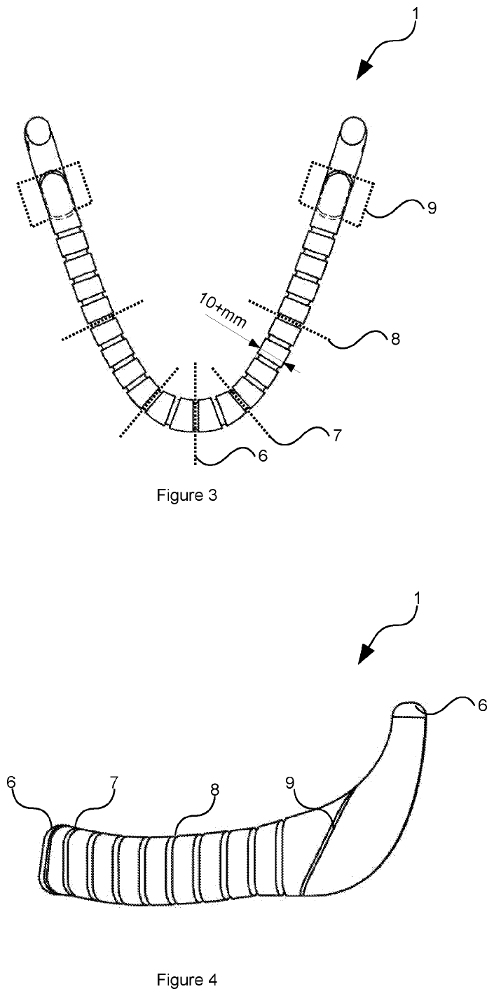

With reference to FIG. 3, a plurality of cutting guides may be provided at anatomically placed resection margins for ease of referencing in use. Specifically, FIG. 3 shows the cutting guides comprising a midline symphysis margin 6, parasymphysis margin 7, mid margin 8 and gonial margin 9 cutting guide.

Whereas various cutting guides 4, such as the midline symphysis 6, parasymphysis 7 and mid cutting guides may be orthogonal, various cutting guide 4 may be angled such as the gonial angle cutting guide 9 as a substantially provided in FIG. 4.

In the embodiment shown in FIG. 3 additional cutting guides may be provided which, in embodiments, may be marked or coloured differently. In the embodiment shown in FIG. 3, the spacer 1 may comprise up to 23 cutting guides 4.

The spacer 1 is rigid in that it is relatively stiff and does not bend easily such that a section cut therefrom in the manner described below may be a structural mandible component to preserve the occlusion and surrounding soft tissues in their correct anatomical positions, reducing or eliminating scarring and/or malocclusion. With reference to FIG. 2, the body portion 2 may comprise a height of between 4-5 cm (for application without teeth) and, with reference to FIG. 3, a minimum thickness of 10 mm in embodiments. As alluded to above, the spacer 1 may be made from a polymer such as PEEK which, given these exemplary dimensions, provides adequate rigidity/stiffness. Furthermore, the cutting guides 4 may be configured to not substantially intrude into the spacer such as by being to a depth of less than approximately 1 mm.

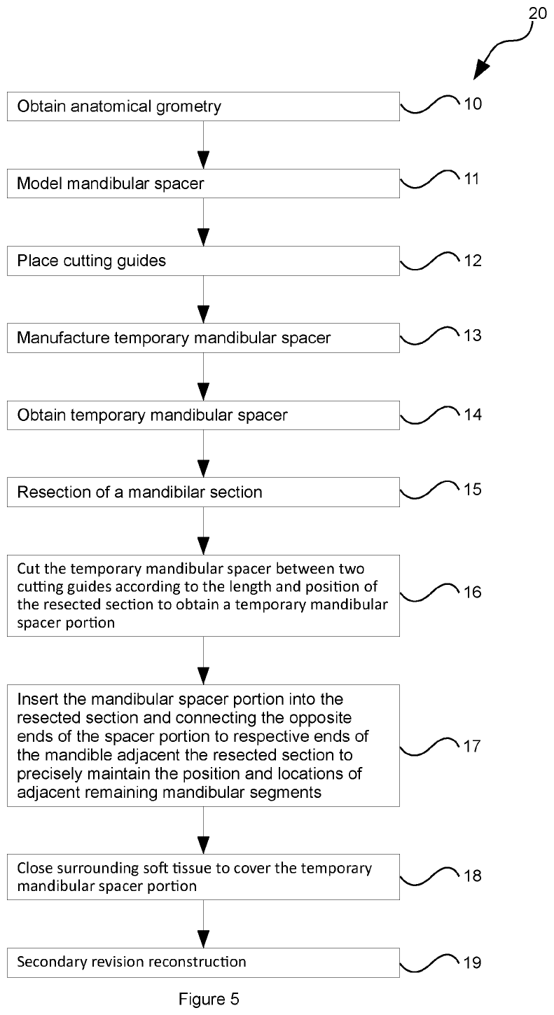

FIG. 5 illustrates a surgical procedure 20 for mandibular reconstruction. The procedure 20 may comprise obtaining anatomical geometry at step 10 from which the spacer 1 is modelled at step 11. In embodiments, obtaining anatomical geometry 10 may comprise obtaining CAT scan survey data from a plurality of patients, such as from 100 patients or more, and deriving various average geometries therefrom for the purposes of modelling a plurality of spacers 1 of differing sizes. As alluded to above these plurality of spacers 1 may be provided pre-packaged and sterilised for selection from according to the sizing of the patient during surgery. In embodiments, spacers 1 may be sized and classified according to gender, ethnicity, age and the like.

Step 12 may comprise locating cutting guides 4 with reference to the model of the spacer 1. As alluded to above, the cutting guides 4 may be placed at anatomical margins of the mandible 6-9 as outlined above.

The procedure 20 may comprise the manufacture of the spacer at step 13.

As such, during the primary mandibular reconstruction surgery, the surgeon may obtain a temporary mandibular spacer 1 at step 14. As outlined above, the surgeon may select spacer 1 from a plurality of prepackaged and sterilised spacers according to the sizing of the patient and, in embodiments, according to the age, gender and ethnicity of the patient.

Step 15 may comprise the resection of the mandible section according to the size and position of the diseased portion therein.

At step 16, the surgeon may cut the temporary mandibular spacer 1 with a reciprocating saw or the like and the length and position of the resected section of the mandible to obtain a temporomandibular spacer portion 16. The cuts may be made with reference to the cutting guides 4.

Step 17 comprises inserting the cut temporary mandibular spacer portion into the resected section and interfacing/connecting the opposite ends of the spacer portion to respective ends of the mandible adjacent the resected section to more precisely maintain the position and locations of the adjacent remaining mandibular segments.

The cutting guides 4 allow for the more accurate shaping and sizing of the temporary mandible of spacer portion according to the resected section including for allowing the opposite ends thereof to conform more accurately with the adjacent bone portions of the resected section to increase structural rigidity of the interface and therefore the mandible as a whole.

Step 18 may comprise closing the surrounding soft tissue to cover the temporomandibular spacer portion.

The temporary mandibular spacer portion may be then left in place such as for more than 12 months for example.

Should the patient survive, the procedure 20 may comprise secondary revision reconstruction 19 wherein the temporary mandibular spacer portion is removed and replaced with a more permanent implant such as autogenous implant.

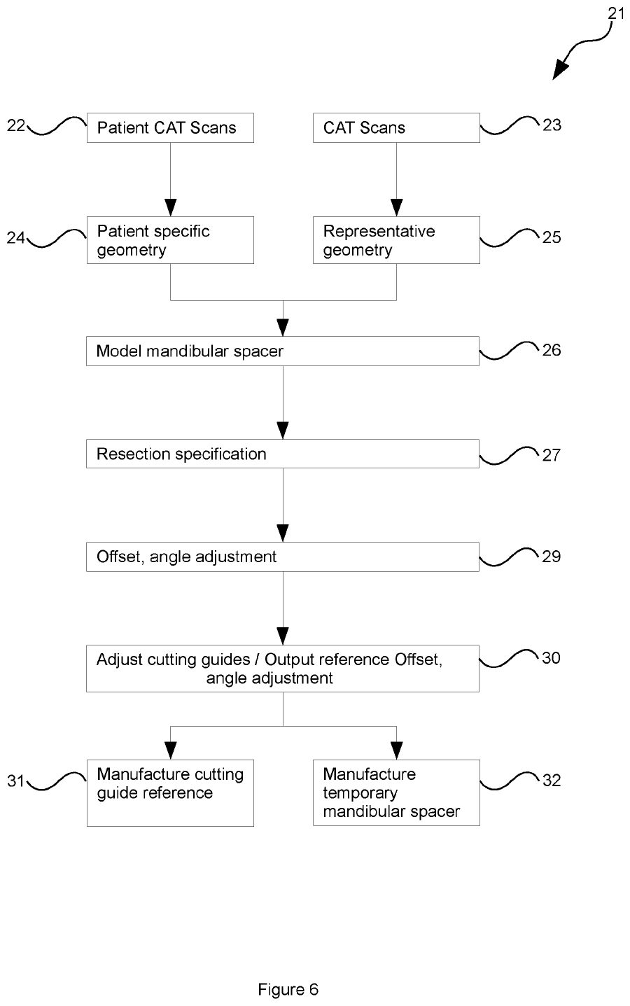

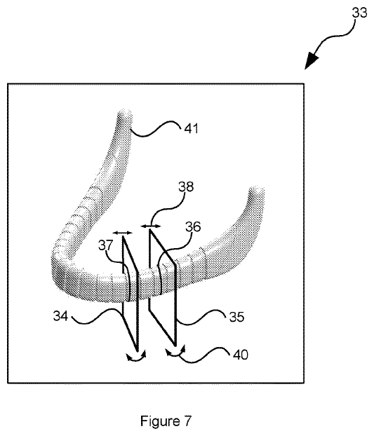

FIG. 6 illustrates the method 21 adjustment for more accurately the cutting of the temporary mandibular spacer portion.

As alluded to above, the method 21 may comprise obtaining CAT scan or the like survey data at step 23 from a plurality of patients so as to obtain representative geometry therefrom 25 for the purposes of manufacturing a plurality of spacers 1 prepackaged and sterilised for selection during surgery. In alternative embodiments the spacer 1 may be manufactured patient specifically wherein patient CAT scans are obtained at step 22 and from which patient specific geometry 24 is derived at step 24 for the purposes of manufacturing a patient specific mandibular spacer 1 which may be produced by a rapid manufacture process such as 3D printing.

Step 26 may comprise modelling the mandibular spacer 1 such as using computer aided modelling.

Step 27 may comprise pre-surgery preparation resection specification for at least one of offset and/or angle adjustment at step 29.

FIG. 7 shows an exemplary computer aided modelling interface 33 for the configuration of the offset and/or angle adjustments. The interface 33 may comprise a computer model 41 of the mandible derived from the patient specific geometry 24 or the representative geometry 25. Layers may be overlaid the model 41 to visualise the location of the disease, including those obtained from imaging such as CAT scans.

Step 29 may comprise the configuration of the offset and/or angle adjustments wherein the interface 33 may comprise first and second cutting plane controls 34, 35 each having associated offset adjustment controls 38 and angle adjustment controls 40 for the adjustment of the location and angle of the cutting plane control 34, 35 to configure the respective planar intersection cuts 37, 36.

Step 32 may comprise the manufacture of the temporary mandibular spacer with adjusted cutting guides 4. For example, the spacer 1 may be manufactured from a rapid manufacturing process such as 3D printing comprising the integrally formed cutting guides and wherein the cutting guides 4 are configured using the offset and/or angle adjustments.

Alternatively, at step 30, offset and angle adjustments may be output which may be used by the surgeon to make cuts with reference to the existing cutting guides 4. For example, the surgeon may measure 3 mm in from the third cutting guide 4 from the midline symphysis margin 6.

In alternative embodiments, a guide reference may be printed or produced which is overlaid the spacer 1. For example, a printed paper reference guide may be output from a printer which is overlaid the surface of the spacer 1 with reference to the existing cutting guides. The reference guide may comprise a cutting guide reference and cutting guideline which may be used to make a marking on the spacer 1 for cutting the spacer.

Alternatively, a reference guide jig may be produced from plastic such as from a 3D printing process which may overlay the spacer 1 and reference the geometry, existing cutting guides or location holes (not shown) of the spacer 1 to locate the reference guide jig with reference to the spacer 1. The reference guide jig may comprise a cutting face along which a mark is made or the reciprocating saw or the other like is guided to cut the spacer 1 more accurately. Once cut, the reference guide jig may be removed and discarded.

In embodiments, bone plates and screws may be utilised to fasten the respective ends of the mandible adjacent the resection to the temporary mandibular spacer portion.

The surrounding soft tissue may then be closed off to cover the implanted temporary mandibular spacer portion.

Thereafter, the spacer portion may be left in place for a substantial time, such as more than 4 months but typically between 6 and 12 months while the prognosis of the patient is monitored including while radiotherapy or chemotherapy is administered and, if the patient prognosis is favourable, planning of secondary revision surgery.

The secondary revision reconstruction may comprise opening the surrounding soft tissue, removal of the temporary mandibular spacer portion from the tissue bed and performing secondary/definitive mandibular reconstruction wherein the temporary mandibular spacer portion may be replaced with autogenous vascularized bone or other alloplastic material for a more permanent reconstruction.

If further cancerous growth is detected, revision surgery may widen the resection including in temporally implanting a further wider temporary mandibular spacer portion.

The foregoing generic description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to best explain the principles of the invention and its practical applications, they thereby enable others skilled in the art to best utilize the invention and various embodiments with various modifications as are suited to the particular use contemplated. It is intended that the following claims and their equivalents define the scope of the invention.

1. A surgical procedure for cancerous mandibular reconstruction, the procedure comprising:
a primary resection and temporary reconstruction phase comprising:
obtaining a temporary mandibular spacer, the spacer having a rigid, biocompatible and radiolucent polymer material body being of a generic and whole mandibular shape;
resection of a cancerous section from a mandible of a patient;
cutting the temporary mandibular spacer according to the length and position of the resected section to obtain a temporary mandibular spacer portion;
inserting the mandibular spacer portion into the resected section and interfacing the opposite ends of the spacer portion to respective ends of the mandible adjacent the resected section to more precisely maintain the position and locations of adjacent remaining mandibular segments;
closing surrounding soft tissue to cover the temporary mandibular spacer portion; and
a secondary revision reconstruction phase performed a substantial time after the primary resection and temporary reconstruction phase, the revision reconstruction phase comprising:
opening the surrounding soft tissue;
removal of the temporary mandibular spacer portion; and
secondary mandibular reconstruction.

2. The surgical procedure as claimed in claim 1, wherein the whole mandibular shape comprises an elongate body portion curved through a midline symphysis location and terminating at posterior upwardly extending ramus process portions.

3. The surgical procedure as claimed in claim 2, wherein the upwardly extending ramus process portions terminate in superior condylar process portions.

4. The surgical procedure as claimed in claim 1, wherein the temporary mandibular spacer comprises a plurality of cutting guides and wherein cutting the temporary mandibular spacer comprises cutting the mandibular spacer with reference to the cutting guides.

5. The surgical procedure as claimed in claim 4, wherein the cutting guides comprise at least one cutting guide being orthogonal with an elongate axis of the elongate body portion.

6. The surgical procedure as claimed in claim 4, wherein the cutting guides comprise at least one cutting guide being angled at an angle with respect to an elongate axis of the elongate body portion.

7. The surgical procedure as claimed in claim 6, wherein the angle is angled with reference to the gonial angle.

8. The surgical procedure as claimed in claim 4, wherein the cutting guides are planar intersection cutting guides.

9. The surgical procedure as claimed in claim 4, wherein the cutting guides are located according to anatomically placed resection margins comprising at least one of a midline symphysis, parasymphysis and tooth location margins.

10. The surgical procedure as claimed in claim 4, wherein the cutting guides are scored into a surface of the spacer to a depth of less than 2 mm.

11. The surgical procedure as claimed in claim 10, wherein the cutting guides have a width of approximately 2 mm.

12. The surgical procedure as claimed in claim 1, wherein the secondary mandibular reconstruction comprises mandibular reconstruction comprising insertion of at least one of autogenous vascularized bone and alloplastic material.

13. The surgical procedure as claimed in claim 1, wherein the secondary revision reconstruction phase is performed more than 4 months after the primary resection and temporary reconstruction phase.

14. The surgical procedure as claimed in claim 1 this, wherein the secondary revision reconstruction phase is performed more than 6 months after the primary resection and temporary reconstruction phase.

15. The surgical procedure as claimed in claim 1, wherein the secondary revision reconstruction phase is performed more than 12 months after the primary resection and temporary reconstruction phase.

16. The surgical procedure as claimed in claim 1, wherein the material is autoclave compatible.

17. The surgical procedure as claimed in claim 1, wherein the material is a polymer.

18. The surgical procedure as claimed in claim 17, wherein the material comprises Polyether ether ketone (PEEK).

19. The surgical procedure as claimed in claim 1, wherein the method comprises providing a plurality of generically shaped spacers of different sizes and wherein obtaining the spacer comprises selection of an appropriate size according to patient sizing.

20. The surgical procedure as claimed in claim 19, wherein the generic mandibular shape is derived from a human mandibles anatomical survey.

21. The surgical procedure as claimed in claim 20, wherein the generically shaped spacers are each classified according to at least one of age, gender and ethnicity according to the anatomical survey.

22. The surgical procedure as claimed in claim 1, wherein connecting the opposite ends of the spacer portion to the respective ends of the resected mandible comprises bone plate and screw affixation.

23. The surgical procedure as claimed in claim 1, wherein the procedure further comprises radiation therapy prior the secondary revision reconstruction phase.

24. The surgical procedure as claimed in claim 4, wherein the procedure further comprises cutting the temporary mandibular spacer further according to at least one of offset and angle adjustments.

25. The surgical procedure as claimed in claim 24, wherein the least one of offset and angle adjustments are configured with reference to at least of size and position of a diseased portion of the mandible.

26. The surgical procedure as claimed in claim 24, wherein the procedure further comprises computer aided modelling comprising display of a model of the temporary mandibular spacer and user interface control thereon for configuration of the at least one of the offset and the angle adjustments.

27. The surgical procedure as claimed in claim 26, wherein the user interface controls comprise at least one of offset and angle adjustment controls.

28. The surgical procedure as claimed in claim 24, wherein the further comprises forming the cutting guides on the spacer with reference to the at least one of the offset and angle adjustments.

29. The surgical procedure as claimed in claim 24, further comprises cutting the temporary mandibular spacer with reference to a cutting guide and the at least one of the offset and angle adjustments.

30. The surgical procedure as claimed in claim 24, wherein the procedure further comprises making a cutting reference which is overlaid the spacer and which is configured according to the at least one of the offset and angle adjustments.

31. The surgical procedure as claimed in claim 30, wherein the cutting guide reference references at least one of geometry, cutting guides and reference engagements of the spacer.

32. The surgical procedure as claimed in claim 30, wherein the cutting guide reference comprises a cutting guide face for guiding a cutting tool.

33. A temporary mandibular spacer, the spacer having a rigid, biocompatible and radiolucent polymer material being of generic and whole mandibular shape.

34. A temporary mandibular spacer as claimed in claim 33, wherein the whole mandibular shape comprises an elongate body portion curved through a midline symphysis location and terminating at posterior upwardly extending ramus process portions.

35. A temporary mandibular spacer as claimed in claim 33, further comprising a plurality of cutting guides.

36. A temporary mandibular spacer as claimed in claim 35, wherein the cutting guides are planar intersection cutting guides.

37. A temporary mandibular spacer as claimed in claim 35, wherein the cutting guides are located at anatomically placed resection margins.

38. A temporary mandibular spacer as claimed in claim 37, wherein the resection margins comprise at least one of midline symphysis, parasymphysis and gonial angle margins.

39. A temporary mandibular spacer as claimed in claim 33, wherein the temporary mandibular spacer is obtained from anatomical survey data obtained from a plurality of patients.

40. A temporary mandibular spacer as claimed in claim 39, wherein the temporary mandibular spacer is classified according to at least one of age, gender and ethnicity according to the anatomical survey data.
